(19) United States
(12) Patent Application Publication
(10) Pub. No.: US 2009/0186953 A1
(43) Pub. Date: Jul. 23, 2009

(54) OXIDE MIXTURE

(76) Inventors: Ulrich Gesenhues, Moers (DE); Bernd Proft, Neukirchen-Vluyn (DE); Elke Hirschberg, Moers (DE)

Correspondence Address: FULBRIGHT & JAWORSKI, LLP, 666 FIFTH AVE, NEW YORK, NY, 10103-3198, US

(21) Appl. No.: 12/162,392

(22) PCT Filed: Feb. 5, 2007
(86) PCT No.: PCT/EP07/51069

§ 371 Date: Jul. 28, 2008

(30) Foreign Application Priority Data
Feb. 3, 2006 (DE) 10 2006 005 484.9

(51) Int. Cl. B01J 21/04; B01J 21/06; C10G 47/04; C10G 11/04; C07C 5/10; C07C 5/25; C07C 51/16; C07C 27/10; C07D 301/03; C07C 27/06

(52) U.S. Cl. 518/728; 502/351; 208/112; 208/123; 585/266; 585/670; 562/548; 562/545; 562/544; 568/700; 549/523; 549/518

(57) ABSTRACT

A catalytically active oxide mixture as well as a process for the production thereof and the use thereof.

The present invention provides a catalytically active oxide mixture as well as a process for the production thereof and the use thereof.

In particular, the present invention concerns porous, acidic catalysts and catalyst supports which contain at least one solid solution of Al₂O₃ and TiO₂ in one another. These catalysts and catalyst supports preferably contain pores which preferably possess a uniform pore size with average diameters d₅₀ of 2 to 40 nm, preferably a uniform pore size with average diameters d₅₀ of 1 to 2 nm.

Pores which possess a pore size with diameters of 2 to 50 nm are also called mesopores; pores of less than 2 nm are also called micropores. The term uniform pore size means that the logarithmic diameter distribution has a width σ of no more than 0.50.

The term pore volume within the meaning of the invention is particularly understood as the contributions of meso- and micropores. Macropores, as are required in shaped catalysts instead of wash-coat layers on metal and ceramic supports, can be subsequently imprinted on previously prepared micro- and mesoporous starting materials using various manufacturing methods according to the prior art, for example by granulating or extruding.

The acidity of the oxide mixture is to be understood as both the Brönsted and the Lewis acidity of the solid surface. On the basis of experimental experience with Al₂O₃ doped TiO₂ white pigments (U. Gesenhues, Chem. Eng. Technol. 24 (2001) 685-694), it is assumed here that surfaces of TiO₂ with Al₂O₃ dissolved therein, where Ti is partially substituted by Al in the positions for the metal in the solid, like pure TiO₂ in the hydroxylated state, i.e. for example in a dry gas atmosphere up to approx. 400° C., are Brönsted acids. Dehydroxylated, i.e. for example in a dry gas atmosphere at higher temperatures, they are Lewis acids.

Mixture in the narrower sense is to be understood as a mix of two or more chemical components present in a spatially separated form. In the broader sense, mixture is also to be understood as the solution of two or more components in one another.

In a solid solution, the components are blended together in atomic or molecular dispersion. Solid solutions may be both crystalline and amorphous and, even with the same substances dissolved in one another, may vary in the content of these substances.

Within the meaning of the invention, the term oxide mixture is to be understood as a mixture in the narrower sense, but which can contain one or more solid solutions.

Doping is intended to mean the atomically and/or molecularly disperse and structurally defined incorporation of small amounts of one component in the volume of a second component (also referred to in this case as the matrix or host lattice) to make specific changes to the properties of the second component; in the case of fine powders this includes incorporation into the surface layer or deposition thereon.

The mixing ratio of components in solid solutions and dopings is meaningfully given chemically in mole % instead of % by weight (wt. %). Thus the composition of the test batches according to the invention, which are intended to give solutions of Al₂O₃ and TiO₂ in one another, and of the catalytically active oxide mixtures of Al₂O₃ and TiO₂ according to the invention is given in mole % Al₂O₃. The difference to 100 mole % corresponds to the proportion of TiO₂ in this case. Since ionic metal oxides usually consist of a dense packing of oxygen anions, in the interstices of which the metal ions are intercalated and often replace one another there, the composition of the test batches can also be given in mole % Al for the sake of simplicity, corresponding to the mole fraction of Al ions. In the binary mixture Al₂O₃/TiO₂, the difference to 100 mole % corresponds in this case to the mole fraction of Ti or of Ti ions, based here in each case on the total amount of substance of the metal ions. The oxide of the metal represented by more than 50 mole % in the mixture usually forms the host lattice and the oxide of the other metal the doping.

These observations may be extended from binary to multi-component systems, for example if the catalytically active oxide mixture of Al₂O₃ and TiO₂ is doped with other metal oxides.

In an aqueous medium, depending on the metal, its oxide or the hydrated preforms thereof can be precipitated as compounds with defined stoichiometry.

For example, in the case of Al, Al(OH)₃ and AlOOH precipitate. Whereas in the case of Ti, according to the traditional view, titanium oxide hydrate or metatitanic acid TiO(OH)₂ precipitates, more recent results show that, in fact, this consists predominantly of nanocrystalline anatase with amorphous TiO₂ as the remainder. Furthermore, in the case of Al, metal oxide and hydrated preforms can be transformed into one another pseudomorphically (B. C. Lippens and J. H. de Boer, Acta Cryst. 17 (1964) 1312). Hereinafter, therefore, the oxide and the hydrated preform are not always differentiated.

Acidity and uniform pore size are two elementary concepts in catalyst development. Thus, it was recognised early on that for certain reactions in petroleum processing, for example cracking, the catalyst surface should be acidic (W. H. Meijs, Diss. Univ. Delft 1962; P. A. Lawrance, GB1010834, 1965). However, for applications in the fluid-catalytic cracking process (fluid-catalytic cracking=FCC) in the petroleum industry, for example pure Al₂O₃ catalysts have too much Lewis acidity, which leads to undesirable hydride abstraction and carbonisation (US-A2003136707, 2003). Very early on, therefore, attempts were made to optimise the acidity by producing a fine-particle mix of Al₂O₃ and TiO₂. In the case of pure TiO₂, the metal atom remains more highly coordinated with O atoms at elevated temperatures, even on the surface, than is the case with pure Al₂O₃. Moreover, TiO₂ adsorbs water more strongly than Al₂O₃, both of which must lead to lower Lewis acidity and the desired higher Brönsted acidity with an ideal solid surface. Furthermore, TiO₂ has higher thermal stability than Al₂O₃ to reaction with H₂O, which is formed during petroleum processing with catalysts, and sulfur compounds, as occur for example in petroleum. This is important because the two reaction products, metal oxide hydrate and metal sulfate respectively, are no longer catalytically active. However, if TiO₂ is dissolved in Al₂O₃ or Al₂O₃ in TiO₂, the metal of the host oxide being partially substituted by the metal of the doping oxide at the positions in the solid, the positions of the doping metal should form new Brönsted and Lewis acid centres on the ideal surface of the solid according to Tanabe's calculation model (K. Tanabe, T. Sumiyoshi, K. Shibata, T. Kiyoura and J. Kitagawa, Bull. Chem. Soc. Japan 47 (1974) 1064-1066). At that time, however, no methods were yet available for the independent quantitative determination of acidity, e.g. using temperature-programmed NH₃ desorption and IR spectroscopy of adsorbed pyridine. Furthermore, the degree of mixing of the two oxides achieved could not yet be determined experimentally, e.g. by means of electron microscopy, ESCA or X-ray diffractometry (XRD), down to atomic or molecular dimensions. Logically, it was also impossible to recognise that the detail of the progress of the precipitation reaction with which the preform of the oxide mixture or of the doped oxide is produced can have a decisive influence on the structure and properties of the product. The description of the production route is therefore frequently disclosed incompletely in the prior art.

Materials with Al₂O₃ as the main component and TiO₂ as the secondary component are known from the prior art—mainly as constituents of complete catalyst systems. Thus, U.S. Pat. No. 3,016,346 discloses mixtures with a maximum of 5.0 wt. % TiO₂. GB943238 discloses mixtures with a maximum of 25 wt. % SiO₂ and TiO₂. GB1010834 discloses mixtures with 1 to 25 wt. % TiO₂. U.S. Pat. No. 3,758,600 discloses mixtures with up to 60 wt. % TiO₂. U.S. Pat. No. 4,465,790 discloses mixtures with 6-8 wt. % TiO₂. WO-A-2004029179 discloses a completely X-ray amorphous oxide mixture of an Al₂O₃ matrix with other metal oxides MeOₓ (metal=Me) uniformly “dispersed” therein, having a molar ratio of Al/Me>5. U.S. Pat. No. 5,229,347 and U.S. Pat. No. 5,558,766 disclose mixtures with 30 wt. % TiO₂, wherein the optimum catalyst acidity is said to lie at a molar ratio of Ti/Al=1/9. Here too, for the first time, a substitution of Al by Ti in the oxide matrix is targeted. However, a solid solution of TiO₂ in Al₂O₃ is actually only thermodynamically stable in the lower percentage range and at temperatures over 1200° C. On the other hand, DE-A-10352816 shows that Al₂O₃, in a fine mixture with TiO₂, produced by controlled precipitation of titanium oxide hydrate and Al(OH)₃ or deposition precipitation of titanium oxide hydrate on Al₂O₃ and subsequent calcining or hydrothermal treatment (hydrothermal=HT), can stabilise the specific surface area and the anatase modification of TiO₂, which is seen as particularly active, at high temperatures, for example in power station and car exhaust catalysis.

In contrast to the Al₂O₃-rich TiO₂—Al₂O₃ mixtures described above, the prior art is less extensive regarding TiO₂—Al₂O₃ mixtures which are rich in TiO₂:

DE-A-10352816 discloses catalysts and catalyst supports of TiO₂—Al₂O₃ mixtures with up to 87 mole % TiO₂; however, these mixtures are precipitates and/or deposition precipitates of TiO₂ on Al₂O₃, with Al₂O₃ or an Al₂O₃ precursor already present in the form of finely dispersed particles. The precipitation product is therefore not present in the form of a solid solution of Al₂O₃ and TiO₂ in one another, either completely or in parts.

EP-A-0517136 discloses catalyst supports with 30 to 70 wt. % TiO₂ and the production thereof, wherein Al and Ti are distributed unevenly over domains of about 5 μm in size.

EP-A-0798362 discloses catalyst particles of TiO₂ with preferably more than 50 wt. % Al₂O₃ or other metal oxides to increase the temperature stability and the specific surface area of the TiO₂. In this document, the various known aqueous methods of producing mixtures of TiO₂ and other oxides such as Al₂O₃, e.g. by co-precipitation, consecutive precipitation, impregnation and compounding are regarded as equivalent. In the application for the FCC process they display no differences even according to the inventors' tests. No information is given regarding the specific surface area, pore volume and other properties of the products. The second oxide causes an increase in the temperature stability of the catalyst material by forming barrier layers on TiO₂; an incorporation into the TiO₂ crystal lattice would destabilise this (DE-A-10352816; U. Gesenhues, Chem. Eng. Technol. 24 (2001) 685-694). The second oxide cannot therefore be dissolved in the TiO₂ crystal. The processes for the production of oxide mixtures disclosed in EP-A-0798362 thus do not lead to a solid solution of Al₂O₃ and TiO₂ in one another.

U.S. Pat. No. 5,922,294 discloses TiO₂ as a catalyst support with Al₂O₃ apparently dissolved therein in a proportion of no more than 60 wt. %, produced from the metal alcoholates with calcining. In this process, TiO₂ in the anatase modification with Al₂O₃ apparently dissolved therein is produced by hydrolysis of a joint alcoholic solution of Ti and Al alcoholates by the addition of water and work-up of the precipitation product by calcining. This production process is obvious, because both metals are already present in atomically disperse mixture in the starting solution. In this way, it appears that up to 39 wt. % Al₂O₃ can be dissolved in the anatase crystal lattice. However, such solid solutions of Al₂O₃ in anatase are thermodynamically metastable against decomposition into rutile and a-Al₂O₃, with only 0.6 to 2.0 wt. % Al₂O₃ being stably soluble in the rutile modification (O. Yamaguchi and Y. Mukaida, J. Am. Ceram. Soc. 72 (1989) 330-333). The products disclosed in U.S. Pat. No. 5,922,294 are only characterised porosimetrically in terms of BET. It has also been shown that it is possible to produce by the same synthesis principle products which achieve a total pore volume of a maximum of 0.33 cm³/g. The anatase modification of TiO₂ is stable up to about 850° C.; XRD reflexes of Al₂O₃ cannot be seen up to this temperature; above this temperature, rutile and corundum are formed (J. Kim, K. C. Song, S. Foncillas, S. E. Pratsinis, J. Eur. Ceram. Soc. 21 (2001) 2863-2872; J. Kim, O. Wilhelm and S. E. Pratsinis, J. Am. Ceram. Soc. 84 (2001) 2802-2808). It is also disclosed that this synthesis principle to be refined by adding an organic complexing agent; a product is obtained with 50 mole % Al, remainder Ti, which is X-ray amorphous when uncalcined and displays only anatase reflexes in an X-ray diffractogram when calcined. The product and its derivatives are mainly microporous—at least 74% so according to BET determination; the total pore volume does not exceed 0.31 cm³/g (E. Y. Kaneko, S. H. Pulcinelli, V. Teixeira da Silva, C. V. Santilli, Appl. Catal. A: General 235 (2002) 71-78). According to the prior art, the lack of reflexes of the various Al₂O₃ modifications in a carefully recorded X-ray diffractogram or the even distribution of Al and Ti in electron micrographs are generally regarded as proof that Al₂O₃ is dissolved in anatase.

A review of acidic TiO₂-based catalysts, their structure and their applications is given e.g. by S. Matsuda and A. Kato in Appl. Catal. 8 (1983) 149-165.

The production of catalyst supports of TiO₂ with Al₂O₃ apparently dissolved therein has been disclosed on a number of occasions. The particular case of the production of TiO₂—Al₂O₃ mixtures from aqueous Ti- and Al-containing solutions without organic additives is disclosed only rarely in the prior art, however.

Thus, U.S. Pat. No. 5,229,347 and U.S. Pat. No. 5,558,766 disclose a production process in which the amount of sodium aluminate solution needed for neutralisation is added to a joint solution of aluminium sulfate and TiOCl₂ or TiOSO₄ without any other acid or base. As a result, Al(OH)₃ and metatitanic acid precipitate out more or less finely mixed. The precipitation product is dried and calcined. It is a disadvantage of this process that, because of the high acidity of the TiOCl₂ solution, only Al₂O₃-rich mixtures with TiO₂ can be produced in this way and that, when neutralised with NaAlO₂, Na⁺ ions are generally difficult to wash out of the precipitation product. The X-ray diffractogram of the calcined product is not very meaningful; it shows no crystalline TiO₂, but only γ- or η-Al₂O₃.

Also disclosed is a process in which a TiO₂ sol and an Al₂O₃ sol are produced separately from metal sulfate or nitrate solution by neutralisation with aqueous NH₃ solution and peptising with HNO₃, then mixed, gelled with aqueous NH₃ solution and dried, and possibly also calcined at 400 to 1000° C.; on the one hand mixtures of pure anatase of 5 nm and boehmite, a crystalline AlOOH modification, with a BET surface area of 390 to 535 m²/g are disclosed, and on the other hand TiO₂—Al₂O₃ mixtures with TiO₂ in the anatase modification and Al₂O₃ in an as yet unclarified form as well as a BET surface area of max. 160 to 260 m²/g; the pore volume can be a maximum of 0.34 cm³/g (S. Sivakumar, C. P. Sibu, P. Mukundan, P. Krishna Pillai and K. G. K. Warrier, Mater. Lett. 58 (2004) 2664-2669). The TiO₂—Al₂O₃ mixtures contain 10 to 57 mole % Al; when uncalcined they contain anatase crystallites 4 to 5 nm in size and boehmite, and when calcined only TiO₂ crystallites, with only the anatase modification up to 800° C. This and changes in the anatase lattice constants with the Al₂O₃ content of the calcined mixture are regarded as indications of a solid solution of Al₂O₃ in the anatase crystals; however, the reliability of the determination of the lattice constants is called into question by an unannotated outlier (Ti—Al (0.70) in Table 1) under five measurements and the complete lack of explanations. Moreover, the increasing inhibition of the conversion of anatase to rutile found with rising Al₂O₃ contents in the mixture (FIGS. 1 and 2 and text) rather suggests a surface occupation of pure anatase crystallites with Al₂O₃. The BET surface area of the products is 390 to 535 m²/g when uncalcined, with an outlier again to be seen at Ti—Al (0.70), and falls constantly by calcining, and more slowly with an increasing Al₂O₃ content of the mixture. However, the reliability of the results on pore volume is called into question by mostly negative but unannotated values, which are even given with 4 decimal places for micropores in Table 2. The distribution of the mesopore diameters is unimodal and constantly covers a range from 2 to 17 nm.

The usefulness of the uniform pore size of a catalyst for the additional control of reactions according to the size of the participating molecules became obvious with the introduction of zeolites in the nineteen seventies. Zeolites are a class of various crystalline aluminosilicates and isotypical aluminophosphates, the pores of which are part of the crystal lattice and are therefore of uniform size. The Al/Si or Al/P ratio characterises the crystal structure and therefore the pore width, which is no more than 1.2 nm, but at the same time determines the acidity. Up to the present, zeolites have been used as catalysts or catalyst supports for precious metals, transition metals and alkali metals for the cracking of naphtha and heavy oil, including the FCC process, for the desulfurisation of naphtha and heavy oil, for the conversion/isomerisation of lower-boiling hydrocarbons to increase the octane number of fuels and to obtain starting materials for organic raw materials chemistry. In addition, catalysts of this type can be employed for Fischer-Tropsch synthesis and for the production of organic fine chemicals.

It has been found that, for the aforementioned reactions, in particular mesoporous and/or microporous catalysts and/or catalyst supports with uniform pore size are advantageous, in which by adjusting the pore dimensions of the oxide mixture to the molecule size of the desired products, the product range of the reactions to be catalysed can be influenced.

A review of the production, structure and applications of catalysts with uniform pore size can be found in: F. Schuth, Ber. Bunsenges. Phys. Chem. 99 (1995) 1306-1315; A. Sayari, Chem. Mater. 8 (1996) 1840-1852; E. Höft et al., J. prakt. Chem. 338 (1996) 1-15; J. Y. Ying, C P. Mehnert, M. S. Wong, Angew. Chem. Int. Ed. 38 (1999) 56-77.

It has also been found that mesoporous and/or microporous metal oxides with uniform pore size can, in addition, be used as packing material in chromatographic columns to separate mixtures of substances. This has already been demonstrated with pure TiO₂, as marketed for example by Sachtleben Chemie GmbH under the SACHTOPORE brand, for pharmaceutical products, herbicides, pesticides and organic isomers.

Materials of this type can therefore not only produce the desired products catalytically but can also separate them from the undesirable by-products, so that reactor and separating apparatus can be advantageously combined in one production process, as this is the concept is already utilised economically in reactive distillation (K. Sundmacher and M. Ivanova, Chemie in unserer Zeit 37 (2003) 268).

U.S. Pat. No. 5,334,368 discloses the most common industrial process for the production of these mesoporous catalysts with uniform pore size. In this process, an emulsion is produced from two immiscible liquids, e.g. one aqueous and one organic, the micelle shape and size of which is controlled by the chain length and other properties of an added surfactant or amphiphilic agent as structure-directing additive. Metal and semimetal compounds dissolved in the first or second liquid phase, such as water glass, aluminium nitrate, but mainly alcoholates of the metals and semimetals, are hydrolysed by changing the pH or adding a water-miscible solvent in the case of the metal and semimetal compounds dissolved in water, or adding water in the case of the organic metal and semimetal compounds. The metal hydroxide, aluminosilicate, aluminophosphate or the corresponding compound is then deposited at the micelle interfaces. The organic solvent can be removed after precipitation by drying and the surfactant or the organic additive by extraction or calcining. Calcining is usually also necessary to dehydrate the precipitation products, which occur in hydrated form, thus converting them to a stable form for use in catalysis. The pore structure of the material can collapse during this operation. The removal of the organic compounds from the precipitation product is not always successful, so that the carbon remaining during calcining in the form of polycondensed aromatics or graphite layers on the surface changes the catalytic activity of the material. On TiO₂ surfaces it accelerates the photocatalytic degradation of organic pollutant molecules (C. Lettmann, K. Hildenbrand, H. Kisch, W. Macyk, W. F. Maier, Appl. Catal. B 32 (2001) 215-227), but in applications in petroleum cracking, such carbon deposits on the surface of the materials act as nuclei for undesirable carbonisation. In order to be able to remove the surfactant or the organic additive more readily, instead of calcining, a hydrothermal treatment may be carried out after drying, followed by extraction and repeated drying. These additional process steps make the product more expensive, however. An advantage of the production of mesoporous materials with uniform pore size via metal and semimetal alcoholates is that the product contains no alkali ions. These are harmful for the acidity and thermal stability of the catalyst. However, it is a disadvantage that organometallic and organic reagents are more expensive and more dangerous than aqueous chemicals. In some products produced by this process, such a regular pore arrangement was observed that additional reflexes were found in the low-angle range of the X-ray diffractogram. Similar production processes are disclosed in U.S. Pat. No. 5,718,878, JP-A-2003119024 and U.S. Pat. No. 5,140,050.

Other processes for the production of mesoporous oxides and mixtures thereof are disclosed in the scientific literature (J. Kim, K. C. Song, S. Foncillas, S. E. Pratsinis, J. Eur. Ceram. Soc. 21 (2001) 2863-2872; J. Kim, O. Wilhelm, S. E. Pratsinis, J. Am. Ceram. Soc. 84 (2001) 2802-2808). In these processes, some mesoporous oxides with uniform pore size are also formed by co-precipitation from solutions of metal alcoholates without structure-directing additives. However, no clear technical instructions are disclosed as to how the pore diameter can be adjusted to values of between 2 and 20 nm regardless of the level of doping.

Generally applicable processes for the production of acidic, mesoporous, TiO₂-rich TiO₂—Al₂O₃ mixtures with uniform pore size from aqueous solutions containing Ti and Al without organic compounds have not been described hitherto.

The object of the present invention is to overcome the disadvantages of the prior art.

In particular, it is the object of the present invention to provide a catalytically active, TiO₂-rich oxide mixture which contains solid solutions of Al₂O₃ and TiO₂ in one another. Another object of the invention is to be able to adjust the acidity of the oxide mixture. Another object of the invention is that the oxide mixture should preferably possess mesopores. Another object of the invention is that the oxide mixture should preferably possess micropores. Another object of the invention is that the pores of the oxide mixture should preferably have a uniform pore size. Another object of the invention is that the pores of the oxide mixture should have an adjustable pore size.

At the same time, the process for the production of this oxide mixture according to the invention should render the use of expensive and dangerous organic or organometallic compounds superfluous.

It should be possible to use the oxide mixture according to the invention as a catalyst and/or catalyst support.

In addition, it should be possible to use the oxide mixture according to the invention in systems for substance separation, preferably as a packing material in chromatographic columns.

In addition, it should also be possible to use the oxide mixture according to the invention in an industrial production process in one and the same apparatus, both as a catalyst and/or catalyst support and as a chromatographic solid phase.

According to the invention, the object is surprisingly achieved by the features of the main claim. Preferred embodiments are found in the subclaims.

In particular, the object is achieved according to the invention by a mesoporous, acidic, catalytically active oxide mixture, which contains a solid solution of TiO₂ and Al₂O₃ in one another, preferably of Al₂O₃ in TiO₂, wherein the mesopores preferably possess a uniform pore size.

In particular, the object is achieved according to the invention by a microporous, acidic, catalytically active oxide mixture, which contains a solid solution of TiO₂ and Al₂O₃ in one another, preferably of Al₂O₃ in TiO₂, wherein the micropores possess a diameter d₅₀ of between 1 and 2 nm.

This oxide mixture according to the invention is obtainable by the process according to the invention described below. Surprisingly, it has been found that the oxide mixture according to the invention is obtainable in particular by a slow precipitation. The process according to the invention is generally characterised by the following steps:
The hydrated preforms of TiO₂ and Al₂O₃ are precipitated from the aqueous Ti- and Al-containing solutions by initially charging the one and adding the other solution. During this operation the pH is held at a value of between 4 and 8 or subsequently adjusted to this range with aqueous solutions of alkalis or NH₃. No organic or organometallic compounds are used in this process. Preferably according to the invention, (a) an aqueous Ti solution is added slowly to an initially charged alkaline Al solution with the addition of lye in order to prevent the pH from falling below 4, preferably below 7, or (b) an alkaline Al solution is added slowly to the Ti solution, the pH value subsequently being raised slowly up to a maximum of 8, preferably up to a maximum of 7. The term “slowly” here within the meaning of the invention means that the rate of precipitation is between 30 and 1 g, preferably between 30 and 5 g, particularly preferably between 28 and 5 g of product (calculated as TiO₂+Al₂O₃) per litre of batch volume per hour of precipitation period. As precipitation period in (a) the period of the addition of the Ti solution is understood, and in (b) the period of the addition of the Al solution and the lye for the subsequent increasing of the pH value. Batch volume means the volume of the suspension at the end of the precipitation. The quantitative ratio of TiO₂ and Al₂O₃ in the oxide mixture according to the invention is controlled here via the quantitative ratio of the Ti- and Al-containing solutions added.
The oxide mixture is separated off as a precipitation product, preferably by filtration, and washed, preferably with aqueous (NH₄)₂CO₃ solution, in order to displace Na⁺ ions and SO₄²⁻ ions, since these are a problem in the application of the catalysts and catalyst supports: Na⁺ ions allow the specific surface area to shrink under temperature load and accelerate the conversion of the preferred anatase modification of TiO₂ to rutile; SO₄₂ ions can pass into the product stream in petroleum chemistry, where on the one hand generally low sulfur contents are desired and on the other hand they react with Al₂O₃ to form Al₂(SO₄)₃ and can deactivate the catalyst in this way.
The purified oxide mixture is dried and possibly calcined or HT-treated.

Surprisingly, it has been found that the slow addition according to the invention of an aqueous Ti solution to an aqueous Al solution or vice versa leads to an oxide mixture which, even after calcining or HT treatment, still contains Al₂O₃ and TiO₂ so finely blended together that there is at least in part a solid solution of Al₂O₃ and TiO₂ in one another. Furthermore, it has surprisingly been found that a wide distribution of the pore diameter is obtained if, according to the invention, according to (a) an aqueous Ti solution is added to an aqueous Al solution, and that a uniform pore diameter is obtained, which can be enlarged by calcining or HT treatment from 2 to 3 nm to up to 15 nm if, according to (b), an aqueous Al solution is added to an aqueous Ti solution.

Surprisingly, the degree of chemical mixing as well as the pore size and uniformity of the catalytically active oxide mixture produced according to the invention can be adjusted independently of one another and of the quantitative ratio of TiO₂/Al₂O₃.

In contrast, a precipitation of fine particle size Al(OH)₃ and titanium oxide hydrate consecutively in the same vessel or a simultaneous rapid joint addition of aqueous Ti-containing and Al-containing solutions at a pH in the neutral range gave products which, after calcining or HT treatment, were a mixture of Al₂O₃ and TiO₂ according to XRD and SEM with EDX (DE-A-10352816).

An additional doping of the oxide mixture according to the invention with other metal ions during or after production is possible using methods according to the prior art, e.g. by dissolved metal salts in the TiOSO₄, Al₂(SO₄)₂ or NaAlO₂ solution or by impregnation with metal salt solutions after precipitation and drying (DE-A-10352816). In addition, an occupation of the oxide mixture according to the invention with transition metal oxides, e.g. oxides of V and/or Mo, with precious metals or other VIIIB metals or the sulfides thereof is possible according to the prior art.

The oxide mixture according to the invention can be processed immediately after precipitation, after drying, after calcining or after HT treatment according to the prior art after conventional pretreatment to form granules or shaped bodies (DE-A-19913839).

The catalytically active oxide mixture according to the invention is used, for example, as a catalyst or catalyst support in petrochemicals, basic organic chemicals and olefin polymerisation.

The invention provides in detail:
a catalytically active oxide mixture containing Al₂O₃ and TiO₂;
a catalytically active oxide mixture containing Al₂O₃ and TiO₂, which contains 0.5 to 45 mole %, preferably 1 to 40 mole % Al, particularly preferably 2 to 40 mole % Al;
a catalytically active oxide mixture containing Al₂O₃ and TiO₂, which contains a solid solution of Al₂O₃ and TiO₂ in one another;
a catalytically active oxide mixture containing Al₂O₃ and TiO₂, which possesses mesopores with an average pore diameter d₅₀ of between 2 and 50 nm, preferably between 2 and 40 nm, particularly preferably between 2 and 20 nm;
a catalytically active oxide mixture containing Al₂O₃ and TiO₂, which possesses micropores with an average pore diameter d₅₀ of between 1 and 2 nm;
a catalytically active oxide mixture containing Al₂O₃ and TiO₂, wherein the particle size d₅₀ of the solid solution is from 0.5 to 10, preferably from 1 to 5, particularly preferably from 1.2 to 3.5 and especially preferably 1.5 to 2.5 μm;
a catalytically active oxide mixture containing Al₂O₃ and TiO₂, wherein the particle size d₅₀ in the fine portion of the solid solution is from 0.5 to 10, preferably from 1 to 5, particularly preferably from 1.2 to 3.5 and especially preferably 1.5 to 2.5 μm;
a catalytically active oxide mixture containing Al₂O₃ and TiO₂, wherein the logarithmic diameter distribution of the pores, preferably of the mesopores, has a width a between 0.01 and 0.50, preferably between 0.02 and 0.40, particularly preferably between 0.05 and 0.40;
a catalytically active oxide mixture containing Al₂O₃ and TiO₂, the BET specific surface area of which is between 5 and 800 m²/g, preferably between 30 and 600 m²/g, particularly preferably between 50 and 450 m²/g;
a catalytically active oxide mixture containing Al₂O₃ and TiO₂, the pore volume of which is between 0.02 and 2 cm³/g, preferably 0.05 to 1 cm³/g, particularly preferably 0.05 to 0.7 cm³/g;
a catalytically active oxide mixture containing Al₂O₃ and TiO₂, of which 30 to 99%, preferably 50 to 99%, particularly preferably 70 to 99% of the pore volume is formed by mesopores;
a catalytically active oxide mixture containing Al₂O₃ and TiO₂, of which the pore volume—in so far as can be determined in the context of measuring accuracy—is formed completely by mesopores;
a catalytically active oxide mixture containing Al₂O₃ and TiO₂, wherein the pore size of the mesopores is adjustable;
a catalytically active oxide mixture containing Al₂O₃ and TiO₂, which contains a thermodynamically monophase system of amorphous TiO₂ with Al₂O₃ dissolved therein;
a catalytically active oxide mixture containing Al₂O₃ and TiO₂, which contains a thermodynamically monophase system of anatase crystals with Al₂O₃ dissolved therein;
a catalytically active oxide mixture containing Al₂O₃ and TiO₂, which contains a thermodynamically biphase system in which the anatase crystals are intergrown with an amorphous solid solution of Al₂O₃ and TiO₂ in one another;
a process for the production of a catalytically active oxide mixture containing Al₂O₃ and TiO₂, by precipitation of hydrated preforms of TiO₂ and Al₂O₃ from the aqueous solutions containing Ti and Al without the use of organic or organometallic compounds, wherein either:
   an aqueous Ti solution is added slowly to a precharged alkaline Al solution with the addition of bases, preferably of basic aqueous solutions, particularly preferably of aqueous solutions of LiOH, NaOH, KOH or NH₃, a pH value of 4 to 8, preferably of 6 to 7, being maintained, or
   an alkaline Al solution is added slowly to the Ti solution, the pH value subsequently being slowly increased by the addition of bases, preferably of basic aqueous solutions, particularly preferably aqueous solutions of LiOH, NaOH, KOH or NH₃, to 4 to 8, preferably to 6 to 7;
a process for the production of a catalytically active oxide mixture containing Al₂O₃ and TiO₂, wherein the aqueous Ti solution is an aqueous titanyl chloride, nitrate or sulfate solution which contains between 50 and 400 g/l, preferably between 60 and 300 g/l, particularly preferably between 60 and 250 g/l titanium;
a process for the production of a catalytically active oxide mixture containing Al₂O₃ and TiO₂, wherein the aqueous Al solution is an aqueous alkali aluminate solution which contains between 50 and 350 g/l, preferably between 60 and 300 g/l, particularly preferably between 70 and 250 g/l aluminium;
a process for the production of a catalytically active oxide mixture containing Al₂O₃ and TiO₂, wherein the oxide mixture is separated off as a precipitation product, preferably by filtration;
a process for the production of a catalytically active oxide mixture containing Al₂O₃ and TiO₂, wherein the oxide mixture is washed, preferably with aqueous (NH₄)₂CO₃ solution;
a process for the production of a catalytically active oxide mixture containing Al₂O₃ and TiO₂, wherein the purified oxide mixture is dried;
a process for the production of a catalytically active oxide mixture containing Al₂O₃ and TiO₂, wherein the purified and dried oxide mixture is calcined at temperatures of between 350 and 900° C., preferably between 400 and 850° C., over a period of between 1 and 20 hours, preferably between 2 and 10 hours, particularly preferably between 2 and 6 hours;
a process for the production of a catalytically active oxide mixture containing Al₂O₃ and TiO₂, wherein the unpurified or purified oxide mixture is HT-treated at temperatures of 100 to 250° C. over a period of between 1 and 20 hours, preferably between 2 and 16 hours, particularly preferably between 2 and 6 hours;
the use of a catalytically active oxide mixture containing Al₂O₃ and TiO₂ as a catalyst and/or catalyst support, preferably in catalytic chemical processes, in the petrochemical industry and/or in organic raw materials synthesis, particularly preferably in (hydro)cracking and/or desulfurisation of naphtha and heavy oil, especially preferably in fluid-catalytic cracking (FCC) and deep-catalytic (DCC) processes for high-sulfur raw materials as well as in the HDS (hydrodesulfurisation) process and in the hydrogenation of aromatic components, in the isomerisation and/or dehydrogenation of low-boiling hydrocarbons, especially preferably in petroleum processing to increase the olefin yield as well as in butane and butene isomerisation, in Fischer-Tropsch synthesis, especially preferably for coal liquefaction, and/or in the partial oxidation of saturated and unsaturated hydrocarbons, especially preferably to form acetic acid, acrylic acid, maleic acid, phthalic and terephthalic acid, in the epoxidation of long-chain olefins, terpenes and of cyclohexane, as well as in the hydroxylation of polynuclear aromatics;
the use of a catalytically active oxide mixture containing Al₂O₃ and TiO₂ in systems for material separation, preferably as a packing material in chromatographic columns;
the use of a catalytically active oxide mixture containing Al₂O₃ and TiO₂ both as a catalyst and/or catalyst support and as a chromatographic solid phase in one and the same apparatus in an industrial production process, preferably by the pressure-change adsorption method for selective aromatics separation by adsorption in the production of fuels from petroleum.

The following examples 1 to 6 according to the invention and the comparative examples 1 and 2 are intended to explain the invention in more detail, without limiting it.

In the examples, the following investigations were carried out on the purified and dried filter cake of the oxide mixture according to the invention after precipitation and of the oxide mixture according to the invention after calcining or HT treatment:
chemical analysis for Ti, Al, NH₄, Na and SO₄;
porosimetry with N₂ sorption;
X-ray diffractometry for crystalline compounds present, the ratio of anatase and rutile in the TiO₂ portion of the sample and the Scherrer crystallite size of the TiO₂;
scanning and transmission electron microscopy with investigation of the distribution of Ti and Al using EDX along lines 100 to 200 nm long over the sample and in 5 to 10 nm-sized surface areas of the sample;
adsorption of pyridine and IR spectroscopy,
particle size distribution (abbreviated as PSD) between 1 and 1000 μm with Fraunhofer diffraction.

The porosimetry method was standardised by IUPAC (Pure & Appl. Chem. 57 (1985) 603-619); in the examples described below, the microporosity was evaluated by the t-method and the mesoporosity by the BJH method (E. P. Barrett, L. G. Joyner and P. P. Halenda, J. Amer. Chem. Soc. 73 (1951) 373 ff.). In addition, the BJH evaluation provides information on pore diameter up to 200 to 300 nm. The average width a of the mesopore diameter distribution according to BJH was determined here from plotting the cumulative pore volume against the logarithm of the pore diameter (cumulative frequency curve of the distribution), by reading off the pore diameter d where the pore volume reaches 16, 50 and 84% of the final value, the following equation being applicable: σ₋=In (d₅₀/d₁₆); σ₊=In (d₈₄/d₅₀); σ=(σ₋+σ₊)/2.

X-ray diffractograms were recorded with Cu-Kα radiation by the Bragg reflection method; for the allocation of the reflexes to crystalline compounds, the Powder Diffraction File of the Internat. Center for Diffraction Data, 1999, and for Al oxides the results of R.-S. Zhou and R. L. Snyder were used (Acta Cryst. B47 (1991) 617-630). Transition Al₂O₃ here means all Al₂O₃ modifications except α and θ.

For the adsorption of pyridine and IR spectroscopy, the powders of the oxide mixture according to the invention obtained were pressed to form a pellet and transferred into an IR cell with CaF₂ windows. The sample was dried there in a gas stream, pyridine being adsorbed and then desorbed again by heating to 400° C., with an IR spectrum being recorded every 50° C. The bands between 1400 and 1700 cm⁻¹ indicate molecularly adsorbed pyridine, Brönsted and Lewis acid centres on the sample surface and the strength of the Lewis centres (G. Busca, Phys. Chem. Chem. Phys. 1 (1999) 723-736).

To determine the particle size distribution, the samples were homogenised and comminuted for 30 seconds in a laboratory crushing mill from IKA and then magnetically stirred for 5 minutes (abbreviated as: 5′MS) in a 0.1 to 0.3% solution of an instantised sodium polyphosphate of average chain length with a P₂O₅ content of approx. 64%, e.g. Calgon N neu from BK Ladenburg, or dispersed in an ultrasound bath for an additional 10 minutes (abbreviated as: 5′MS+10′US), and then measured in a HELOS apparatus from Sympatec, Clausthal with a 633 nm laser beam.

### EXAMPLE 1

### Initial Charge of NaAlO₂ Solution, Addition of TiOSO₄ Solution

To a stirred initial charge of 117 ml of an aqueous NaAlO₂ solution with an Al content of 132 g/l, corresponding to a content of 250 g/l Al₂O₃, 1250 ml TiOSO₄ solution, containing 66 g/l Ti, corresponding to 110 g/l TiO₂, and stabilised with 225 g/l H₂SO₄, are added at a uniform rate of 10 ml per minute. The quantitative ratios correspond to a composition of the oxide mixture according to the invention of 25 mole % Al, remainder Ti. When a pH of 7 is reached in the initial charge, 10% sodium hydroxide solution is then pumped in simultaneously in such a way that this pH value is maintained. On completion of the addition, the volume is 4.3 l. The precipitation rate according to these data is 18.6 g per l per hour. Stirring is then continued for 1 hour, the mixture is filtered by suction and washed with 24 l (NH₄)₂CO₃ solution per kg of the sum of TiO₂ and Al₂O₃ in the batch, the solution having a concentration of 10 g/l. The oxide mixture according to the invention is dried. Samples of 10 g of the oxide mixture are calcined in a muffle-type furnace for 4 hours at 450, 600 and 800° C. and then investigated.

The results are shown in Table 1. An evaluation of the results of the XRD, SEM and EDX analyses shows that, in the oxide mixture according to the invention after calcining, Al and Ti are so finely blended together that a solid solution of TiO₂ and Al₂O₃ in one another must have formed. Crystalline TiO₂ with Al₂O₃ dissolved therein (thermodynamically monophase system) or a mix of pure crystalline TiO₂ and an amorphous solid solution of TiO₂ and Al₂O₃ in one another (thermodynamically biphase system) or a mixture of the two systems may be present in the oxide mixture according to the invention. The results in Table 1 also show that the oxide mixture according to the invention, uncalcined and calcined, has a specific surface area and porosity which are adequate for catalytic applications and which also have adequate temperature stability.

Oxide mixtures according to the invention with the compositions 40 mole %, 15 mole %, 7 mole % and 3 mole % Al, remainder Ti in each case, are produced and investigated in the same way. The results are shown in Table 7. They display values similar to the oxide mixture according to the invention with 25 mole % Al for specific surface area and pore volume. Both values fall slightly with a rising TiO₂ content and markedly with rising calcining temperature, but the pore volume is always greater than that described in Sivakumar, Sibu, Mukundan et al. 2004. This enlarged pore volume is valuable for catalytic applications. The pore diameter from the BJH evaluation (mesopores and pores up to 200 nm) is always nonuniform, both before and after calcining. Nonuniform means that the cumulative frequency curve of the pore diameter distribution is almost linear between 2 and 200 nm, i.e. no pore diameter occurs significantly more frequently than any others. Al and Ti are always blended together just as finely as previously according to SEM and EDX, and XRD shows no reflexes for all TiO₂—Al₂O₃ mixtures before calcining and for Al₂O₃-rich mixtures even after calcining at low temperature, and only anatase reflexes thereafter. Only in the mixture richest in TiO₂ are rutile reflexes also observed at the highest calcining temperature.

The particle size distributions (abbreviated as PSD) of the calcined samples were always bimodal; after 5′MS the maximum particle size was approx. 200 μm, and after 5′MS+10′US approx. 50 μm. In Tables 1 and 7, the quantity of the fine portion is given; that of the coarse portion corresponds to the difference to 100%. In addition, the d₅₀ values of the fine and coarse portions are given. The results show that the primary particles from the precipitation have always aggregated to approx. 2 μm after calcining, regardless of the Al₂O₃ portion, calcining temperature and dispersing intensity and have further agglomerated to 25 to 55 μm (after 5′MS) or 10 to 20 μm (after 5′MS+10′US). The fine portion increases with rising Al₂O₃ content of the samples, but does not decrease with rising calcining temperature, which is advantageous.

### EXAMPLE 2

### Initial Charge of TiOSO₄ Solution, Addition of NaAlO₂ Solution, Neutralisation with Sodium Hydroxide Solution, Calcining

Into a stirred initial charge of 1250 ml TiOSO₄ solution, 117 ml NaAlO₂ solution are pumped at 5 ml per minute; both solutions correspond to those in example 1. The quantitative ratios correspond to a composition of the oxide mixture according to the invention of 25 mole % Al, remainder Ti. Thereafter, the pH is less than 2. Next, 10% sodium hydroxide solution is pumped in at 20 ml per minute up to a pH of 2.5 and at 10 ml per minute up to a pH of 7. In total, 2930 ml are needed. The volume of the batch is then 4.3 l; the rate of precipitation is 10.5 g per l per hour. The oxide mixture according to the invention is subsequently stirred, filtered, washed, dried, calcined and investigated as in example 1.

The results are shown in Table 1. According to XRD, SEM and EDX, after calcining Al and Ti are so finely blended together that a solid solution of TiO₂ and Al₂O₃ in one another must have formed. At a high temperature, this decomposes into the pure oxides. The results in Table 1 also show that the material, when uncalcined and weakly calcined, has an adequate specific surface area and porosity for catalytic applications. The limited temperature stability is attributable to the high Na content. The high SO₄ content can also be disadvantageous for applications in catalysis. The pore diameter distribution according to BJH is unimodal in the 2 to 200 nm range and lies within the mesopore range; the mesopore diameter is uniform in this material and grows with the calcining temperature. The fact that the products have unimodal mesopores means in particular that there are no pores with diameters of between 50 and 200 nm in the products.

### EXAMPLE 3

### Initial Charge of TiOSO₄ Solution, Addition of NaAlO₂ Solution, Neutralisation with NH₃ Solution, Calcining

The test is carried out as in example 2, but instead of sodium hydroxide solution, 15% aqueous NH₃ solution is added at 5 ml per minute until a pH of 7 is reached. In addition, two sedimentation washes are carried out before filtration, in which the batch is topped up to 60 l with deionised water, left to stand for 4 hours and then 40 l of clear supernatant liquid are siphoned off. On completion of addition of the NH₃ solution, the batch has a volume of 2.3 l; the rate of precipitation in this example is 21.4 g per l per hour.

The results are shown in Table 1. The pore diameter distributions according to BJH correspond to those of the previous example, and the distributions of the mesopore diameters for the precipitation product and the calcining products of the oxide mixture according to the invention are also just as narrow; the distribution width a is given in Table 2 in the column for 25 mole % Al, remainder Ti. The results show, together with SEM and EDX:
the Na content in the product is lower than in the sample according to example 2; as a result, the specific surface area remains more stable at higher temperatures than in the previous samples;
Al and Ti are finely blended together after calcining; a solid solution of TiO₂ and Al₂O₃ has formed, which no longer decomposes into the pure oxides even at a high temperature;
the pore volume comes from mesopores and is reduced by calcining;
the mesopore diameter is uniform at every calcining temperature and can be adjusted between 2 and 15 nm by means of temperature.

Oxide mixtures according to the invention with the compositions 40 mole %, 15 mole %, 7 mole % and 3 mole % Al, remainder Ti in each case, are produced and investigated in the same way. The results are in Table 2. The rutile phase of the TiO₂ and Al₂O₃ modifications is not detectable in the XRD in any oxide mixture according to the invention, even at the highest calcining temperature. The finely dispersed distribution of TiO₂ and Al₂O₃ in one another is guaranteed according to TEM with EDX for Al and Ti along lines from 100 to 200 nm long over the sample and in 5 to 10 nm-sized surface areas of the sample even in the oxide mixture according to the invention with 40 mole % Al after calcining, i.e. the calcined oxide mixtures according to the invention consist either completely of anatase with Al₂O₃ dissolved therein (thermodynamically one phase) or of anatase and an amorphous solution of Al₂O₃ and TiO₂ in one another (thermodynamically two phases). The uniform mesopore size is already present in the precipitation product (Table 2) and is maintained during calcining and can be adjusted almost independently of the Al/Ti ratio of the oxide mixture (Table 2). As in the previous example, none of these products contains any pores with diameters of between 50 and 200 nm.

The oxide mixtures according to the invention according to Table 2 are additionally tested for their Brönsted and Lewis acidity with pyridine adsorption and IR spectroscopy. The samples calcined at 600° C. display almost exclusively Lewis acidity, as expected. The surface concentration of all the Lewis acid centres can be determined from the intensity of the band at 1445 cm⁻¹ after heating in a spectrometer to 100° C.; it falls as the Al₂O₃ content of the samples rises. The fall is attributable to the disappearance of the weakly acidic centres, while the concentration of the strongly acidic centres remains the same, as shown by the intensity of the band at 1445 cm⁻¹ after heating in a spectrometer to 400° C. and the development of the three single bands around 1600 cm⁻¹ on heating. The average acidity of the surface centres thus increases with the Al₂O₃ content of the samples.

The particle size distributions are bimodal with a maximum particle size of approx. 100 μm after 5′MS+10′US. In contrast to example 1, the fine portion is smaller (after 5′MS+10′US), but it also falls with an increasing TiO₂ content of the products.

### EXAMPLE 4

### HT Treatment Instead of Calcining

The test is carried out as in example 3, but the filter cakes of the oxide mixture according to the invention are slurried with deionised water to 8 to 11% solids and the suspensions are hydrothermally treated in a steel autoclave for 2 to 16 hours at 120 to 180° C. The oxide mixture according to the invention is filtered off, dried and investigated.

The results are shown in Tables 3 to 5:
mesopores of uniform size can also be produced with HT treatment instead of calcining, although the diameter distribution of the mesopores is not as narrow in this case as in samples that have been calcined;
the mesopore diameters can also be adjusted with HT treatment in the desired range of 2 to 15 nm;
to adjust the mesopore diameter, temperature and duration of the HT treatment can be used equally;
as in the previous example, there are no pores in the diameter range of 50 to 200 nm;
the solid solution of Al₂O₃ and TiO₂ in one another produced in the precipitation is not quite as resistant to HT treatment as to calcining in the case of high Al₂O₃ contents;
HT treatment produces a greater pore volume than calcining; it does not depend on the Al₂O₃ content and is thermally stable: this is advantageous for the use of the oxide mixture according to the invention as a catalyst or catalyst support when employed under temperature load and in particular for the production of catalysts or catalyst supports with a low Al₂O₃ content;
the particle size distributions are bimodal as in example 3, and the maximum particle size is approx. 50 μm; however, (after 5′MS+10′US) the fine portion is higher and as the Al₂O₃ content falls, the fine portion grows and the average fine particle diameter falls;

### EXAMPLE 5

### Use of a Concentrated TiOSO₄ Solution for Precipitation

The test is carried out as in example 3, but 490 ml TiOSO₄ solution with 280 g/l TiO₂ and 575 g/l H₂SO₄ are initially charged instead of 1250 ml TiOSO₄ solution with 110 g/l TiO₂ and 225 g/l H₂SO₄. In addition, the NH₃ solution is pumped in at only 3 ml per minute. Thereafter, the volume of the batch is 1.5 l. The rate of precipitation is 20 g per l per hour.

The properties of the oxide mixture after drying and calcining are reproduced in Table 6. The results are comparable with those of TiO₂/Al₂O₃ with 25 mole % Al, remainder Ti, in Table 2.

According to this, precipitation with a higher concentration of the TiOSO₄ solution impairs the quality of the oxide mixture only slightly. Precipitations with more highly concentrated solutions permit a higher space-time yield and are therefore economically preferable.

### EXAMPLE 6

### Additional use of a More Concentrated NaAlO₂ Solution

The test is carried out as in example 5, but 89 ml NaAlO₂ solution with 330 g/l Al₂O₃ are pumped in instead of 117 ml NaAlO₂ solution with 250 g/l Al₂O₃. The rate of precipitation is 21 g per l per hour.

The properties of the oxide mixture after drying and calcining are reproduced in Table 6. The results are comparable with those of TiO₂/Al₂O₃ with 25 mole % Al, remainder Ti, in Table 2. According to this, precipitation with a higher concentration of the NaAlO₂ solution impairs the quality of the oxide mixture only slightly.

### COMPARATIVE EXAMPLE 1

### Deposition Precipitation of a TiO₂ Preform on to Previously Prepared, Finely Dispersed Al₂O₃ Particles

In a 6 l round-bottom flask with a KPG stirrer, reflux condenser, contact thermometer and Pilz heating mantle, 1.5 l H₂O and 40 g of a commercial aluminium oxide with a specific surface area of 104 m²/g (pyrogenic, primary particle size approx. 15 nm, non-agglomerated, according to x-ray diffractogram in the 6 modification as per manufacturer's data, in the γ or η but not yet θ modification according to Zhou and Snyder) are initially charged, with stirring. At 90° C., 1710 ml of TiOSO₄ solution, which contains 66 g/l Ti, corresponding to 110 g/l TiO₂, and is stabilised with 225 g/l H₂SO₄, are added at a uniform rate over 180 minutes. The quantitative ratios correspond to a composition of the oxide mixture of 25 mole % Al, remainder Ti. The mixture is then boiled for 30 minutes and subsequently filtered, washed with H₂O and dried. Samples of 10 g of the product are calcined in a muffle type furnace for 4 hours at 450, 600 and 800° C. and investigated.

In the XRD of the calcined products, the intensity ratio of the reflexes of aluminium oxide at 2θ=46° and of anatase at 2θ=48° is always identical, to an accuracy of ±4%, with a mechanically produced mix of the aluminium oxide starting product and a chemically pure, non-surface-modified anatase white pigment according to the sulfate process with 25 mole % Al, remainder Ti. Further results are shown in Table 1. They show that no solution of Al₂O₃ and TiO₂ in one another has formed. No conversion of anatase into rutile is observed.

The example proves that, without molecularly disperse premixing, even if the one component is deposited by precipitation on the particles of the other component in a thin, smooth layer, only mixes of TiO₂ and Al₂O₃ form during calcining.

In addition, the product from this comparative example has such fine particles according to Table 1 (predominantly<1 μm) that it is more difficult to process, for example by the wash-coat process in catalyst production.

### COMPARATIVE EXAMPLE 2

### Deposition Precipitation of an Al₂O₃ Preform on to Particles of a Previously Prepared TiO₂ Preform

In a 6 l round-bottom flask with a KPG stirrer, reflux condenser, contact thermometer and Pilz heating mantle, 2.00 l suspension of a washed but not yet bleached metatitanic acid from the sulfate process for TiO₂ pigments with a calculated TiO₂ content of 356 g/l are initially charged and 498 ml sodium aluminate solution with a calculated Al₂O₃ content of 304 g/l are added within 45 minutes. The quantitative ratios correspond to a composition of the oxide mixture of 25 mole % Al, remainder Ti. On completion of the addition, the pH is 10.7. The mixture is then boiled for 2 hours. Following this, it is neutralised with 20% sulfuric acid within 30 minutes, filtered, washed with H₂O and dried. Samples of 10 g of the product are calcined in a muffle-type furnace for 4 hours at 450, 600 and 800° C. and investigated.

Using SEM and EDX, in addition to the aggregates of the submicrometre-sized primary particles of the metatitanic acid, several micrometre long prisms can be seen in the uncalcined product and angular grains of approx. 1.5 μm in size with an almost smooth surface in the calcined product. Prisms and angular grains contain only Al but no Ti. The XRD results in Table 1 and the SEM investigations show that Al and Ti starting components must already be mixed in a molecularly disperse manner before precipitation so that solid solutions of Al₂O₃ and TiO₂ in one another form. Other results are shown in Table 1.

The particle size distribution of this product (Table 1) is almost unimodal and its average particle size such that it can be applied to shaped catalysts in wash-coat processes without previous grinding.

TABLE 1
Products of TiO₂/Al₂O₃ with 25 mole % Al, remainder Ti, influence
of the choice of starting materials and of the process for
precipitation and washing:

| | | | | |
|---|---|---|---|---|
| Comp. | Comp. | | | |
| example 1 | example 2 | Example 1 | Example 2 | Example 3 |

Table 1a: After drying:

| | | | | | |
|---|---|---|---|---|---|
| % NH₄ | | | | — | 1.5 |
| ppm Na | | 13000 | 2100 | 26000 | 60 |
| % SO₄ | | <0.01 | 0.03 | 4.5 | 0.71 |
| BET (thereof in | 173 | 196 (7) | 424 (279) | 296 (190) | 417 (35) |
| micropores) [m²/g] | | | | | |
| Pore volume | | 0.29 | 0.64 (0.47) | 0.19 (0.07) | 0.36 (0.33) |
| (thereof in | | | | | |
| mesopores | | | | | |
| [cm³/g] | | | | | |
| Mesopore | | None | None | Monomodal | Monomodal |
| diameter [nm] | | preferred | preferred | 3.3 | 2.7 |
| Crystalline | Transition | Anatase, | — | — | — |
| compounds | Al₂O₃ | Al(OH)₃ | (amorphous) | (amorphous) | (amorphous) |
| found | | bayerite | | | |

Table 1b: Calcined at 450° C.:

| | | | | | |
|---|---|---|---|---|---|
| BET (thereof | 121 | 102 | 249 (0) | 157 (0) | 282 (0) |
| in micropores) | | | | | |
| [m²/g] | | | | | |
| Pore volume | | | 0.65 | 0.17 | 0.30 |
| [cm³/g] | | | | | |
| Mesopore | | | Small | Monomodal | Monomodal |
| diameter [nm] | | | preferred | 3.8 | 3.6 |
| Crystalline | Anatase, | Anatase, | — | Anatase | — |
| compounds | transition | AlOOH | (amorphous) | | (amorphous) |
| found | Al₂O₃ | boehmite | | | |
| | | η-Al₂O₃ | | | |
| Anatase | 11 | 12 | | | |
| crystallite size | | | | | |
| [nm] | | | | | |
| PSD after | 93/<1 | 97/3.5 | 40/1.8 | | |
| 5′MS: quantity | | | | | |
| [%]/d₅₀ [μm] | | | | | |
| of fine portion | | | | | |
| PSD after | 22 | Approx. | 42 | | |
| 5′MS: d₅₀ [μm] | | 20 | | | |
| of coarse | | | | | |
| portion | | | | | |
| PSD after | 95/<1 | 97/2.4 | 67/1.9 | | 45/2.2 |
| 5′MS + 10′US: | | | | | |
| quantity [%]/ | | | | | |
| d₅₀ [μm] of | | | | | |
| fine portion | | | | | |
| PSD after | 15 | Approx. | 17 | | 30 |
| 5′MS + 10′US: | | 20 | | | |
| d₅₀ [μm] of | | | | | |
| coarse portion | | | | | |

Table 1c: Calcined at 600° C.:

| | | | | | |
|---|---|---|---|---|---|
| Weight loss | 14.2 | 10.8 | 16.7 | 12.1 | 14.7 |
| [%] | | | | | |
| BET (thereof | 103 | 85 | 98 (0) | 71 (0) | 167 (0) |
| in micropores) | | | | | |
| [m²/g] | | | | | |
| Pore volume | | | 0.48 | 0.15 | 0.24 |
| [cm³/g] | | | | | |
| Mesopore | | | None | Monomodal | Monomodal |
| diameter [nm] | | | preferred | 6.1 | 4.5 |
| Crystalline | Anatase, | Anatase, | Anatase | Anatase | Anatase |
| compounds | transition | η-Al₂O₃ | | | |
| found | Al₂O₃ | | | | |
| Anatase | 15 | 18 | 11 | 9 | 6 |
| crystallite size | | | | | |
| [nm] | | | | | |
| PSD after | 89/<1 | 92/4.2 | | | |
| 5′MS: quantity | | | | | |
| [%]/d₅₀ [μm] | | | | | |
| of fine portion | | | | | |
| PSD after | 22 | 19 | | | |
| 5′MS: d₅₀ [μm] | | | | | |
| of coarse | | | | | |
| portion | | | | | |
| PSD after | 90/<1 | 92/3.8 | | | |
| 5′MS + 10′US: | | | | | |
| quantity [%]/ | | | | | |
| d₅₀ [μm] of | | | | | |
| fine portion | | | | | |
| PSD after | 20 | 16 | | | |
| 5′MS + 10′US: | | | | | |
| d₅₀ [μm] of | | | | | |
| coarse portion | | | | | |

Table 1d: Calcined at 800° C.:

| | | | | | |
|---|---|---|---|---|---|
| BET (thereof | 96 | 57 (0) | 49 (6) | 30 | 68 (0) |
| in micropores) | | | | | |
| [m²/g] | | | | | |
| Pore volume | | 0.25 | 0.36 | — | 0.18 |
| [cm³/g] | | | | | |
| Mesopore | | None | Large | — | Monomodal |
| diameter [nm] | | preferred | preferred | | 7.8 |
| Crystalline | Anatase, | Anatase, | Anatase | Anatase, | Anatase |
| compounds | transition | η-Al₂O₃ | | transition | |
| found | Al₂O₃ | | | Al₂O₃ | |
| Anatase | 24 | 27 | 18 | 23 | 14 |
| crystallite size | | | | | |
| [nm] | | | | | |

TABLE 2
Products of TiO₂/Al₂O₃ of different compositions after the calcining
process (Example 3):

| Mole % Al, remainder Ti | | | | |
|---|---|---|---|---|
| 40 | 25 | 15 | 7 | 3 |

Table 2a: After drying:

| | | | | | |
|---|---|---|---|---|---|
| % NH₄ | 1.2 | 1.5 | 2.0 | 3.1 | 3.4 |
| ppm Na | 80 | 60 | 370 | 170 | 110 |
| % SO₄ | 3.9 | 0.71 | 0.24 | 0.06 | 0.03 |
| BET (thereof | 415 (76) | 417 (35) | 411 (44) | 393 (34) | 356 (26) |
| in micropores) | | | | | |
| [m²/g] | | | | | |
| Pore volume | 0.29 (0.25) | 0.36 (0.33) | 0.28 (0.24) | 0.26 (0.20) | 0.25 (0.20) |
| (thereof in | | | | | |
| mesopores) | | | | | |
| [cm³/g] | | | | | |
| Mesopores: | 2.7/0.38 | 2.7/0.37 | 2.7/0.37 | 2.6/0.36 | 2.7/0.38 |
| d₅₀ [nm]/ | | | | | |
| ±σ [—] | | | | | |
| Crystalline | — | — | — | — | — |
| compounds | (amorphous) | (amorphous) | (amorphous) | (amorphous) | (amorphous) |
| found | | | | | |

Table 2b: Calcined at 450° C.:

| | | | | | |
|---|---|---|---|---|---|
| Weight loss [%] | 15.1 | 13.1 | 14.7 | 13.4 | 10.4 |
| BET (thereof in | 275 (0) | 282 (0) | 232 (0) | 148 (0) | 110 (0) |
| micropores) | | | | | |
| [m²/g] | | | | | |
| Pore volume | 0.30 (0.27) | 0.30 (0.27) | 0.25 (0.24) | 0.19 (0.20) | 0.17 (0.17) |
| (thereof in | | | | | |
| mesopores | | | | | |
| [cm³/g] | | | | | |
| Mesopores: | 3.6/0.09 | 3.6/0.07 | 3.6/0.10 | 4.0/0.12 | 4.7/0.15 |
| d₅₀ [nm]/±σ [—] | | | | | |
| Crystalline | — | — | Anatase | Anatase | Anatase |
| compounds | (amorphous) | (amorphous) | | | |
| found | | | | | |
| Anatase | | | 4 | 7 | 10 |
| crystallite size | | | | | |
| [nm] | | | | | |
| PSD after 5′MS + | 55/2.3 | | | | |
| 10′US: | | | | | |
| quantity [%]/ | | | | | |
| d₅₀ [μm] of fine | | | | | |
| portion | | | | | |
| PSD after 5′MS + | 25 | | | | |
| 10′US: d₅₀ | | | | | |
| [μm] of coarse | | | | | |
| portion | | | | | |

Table 2c: Calcined at 600° C.:

| | | | | | |
|---|---|---|---|---|---|
| Weight loss | 16.8 | 14.7 | 15.9 | 14.4 | 10.7 |
| [%] | | | | | |
| BET (thereof | 185 (0) | 167 (0) | 121 (0) | 96 (0) | 66 (0) |
| in micropores) | | | | | |
| [m²/g] | | | | | |
| Pore volume | 0.26 (0.28) | 0.24 (0.25) | 0.21 (0.22) | 0.17 (0.18) | 0.14 (0.15) |
| (thereof in | | | | | |
| mesopores | | | | | |
| [cm³/g] | | | | | |
| Mesopores: | 4.3/0.12 | 4.5/0.14 | 5.5/0.14 | 5.8/0.15 | 6.3/0.14 |
| d₅₀ [nm]/ | | | | | |
| ±σ [—] | | | | | |
| Crystalline | Anatase | Anatase | Anatase | Anatase | Anatase |
| compounds | | | | | |
| found | | | | | |
| Anatase | 5 | 6 | 7 | 8 | 12 |
| crystallite size | | | | | |
| [nm] | | | | | |
| PSD after | 52/2.5 | 45/2.2 | 44/2.7 | 40/2.5 | 30/3.1 |
| 5′MS + 10′US: | | | | | |
| quantity [%]/ | | | | | |
| d₅₀ [μm] of | | | | | |
| fine portion | | | | | |
| PSD after | 28 | 30 | 27 | 23 | 25 |
| 5′MS + 10′US: | | | | | |
| d₅₀ [μm] of | | | | | |
| coarse portion | | | | | |

Table 2d: Calcined at 800° C.:

| | | | | | |
|---|---|---|---|---|---|
| Weight loss | 19.3 | 15.3 | 16.2 | 14.6 | 10.8 |
| [%] | | | | | |
| BET (thereof | 94 (0) | 68 (0) | 43 (0) | 31 | 17 |
| in micropores) | | | | | |
| [m²/g] | | | | | |
| Pore volume | 0.21 (0.22) | 0.18 (0.18) | 0.15 (0.15) | 0.10 (0.10) | 0.06 (0.06) |
| (thereof in | | | | | |
| mesopores | | | | | |
| [cm³/g] | | | | | |
| Mesopores: | 6.9/0.11 | 7.8/0.11 | 10.5/0.13 | 10.0/0.16 | 8.3/0.21 |
| d₅₀ [nm]/ | | | | | |
| ±σ [—] | | | | | |
| Crystalline | Anatase | Anatase | Anatase | Anatase | Anatase |
| compounds | | | | | |
| found | | | | | |
| Anatase | 10 | 14 | 19 | 21 | 26 |
| crystallite size | | | | | |
| [nm] | | | | | |
| PSD after | 45/3.4 | | | | |
| 5′MS + 10′US: | | | | | |
| quantity [%]/ | | | | | |
| d₅₀ [μm] of | | | | | |
| fine portion | | | | | |
| PSD after | 36 | | | | |
| 5′MS + 10′US: | | | | | |
| d₅₀ [μm] of | | | | | |
| coarse portion | | | | | |

TABLE 3
Products of TiO₂/Al₂O₃ of different compositions after the HT process:

| Mole % Al, remainder Ti | | | | |
|---|---|---|---|---|
| 40 | 25 | 15 | 7 | 3 |

Table 3a: General properties after 4 h HT treatment at 180° C. (Example 4):

| | | | | | |
|---|---|---|---|---|---|
| % NH₄ | 0.29 | 0.21 | 0.18 | 0.21 | 0.14 |
| ppm Na | 120 | 70 | 120 | 90 | 100 |
| % SO₄ | 0.8 | <0.05 | <0.05 | <0.05 | <0.05 |
| BET (thereof | 152 (0) | 135 (0) | 158 (0) | 147 (0) | 114 (0) |
| in micropores) | | | | | |
| [m²/g] | | | | | |
| Pore volume | 0.37 (0.37) | 0.34 (0.34) | 0.37 (0.37) | 0.39 (0.39) | 0.36 (0.36) |
| (thereof in | | | | | |
| mesopores) | | | | | |
| [cm³/g] | | | | | |
| Crystalline | Anatase, | Anatase, | Anatase | Anatase | Anatase |
| compounds | boehmite | a little | | | |
| found | | boehmite | | | |
| Anatase | 8 | 10 | 9 | 9 | 12 |
| crystallite size | | | | | |
| [nm] | | | | | |
| PSD after | 46/3.1 | 50/2.7 | 51/2.0 | 53/1.4 | 49/<1 |
| 5′MS: quantity | | | | | |
| [%]/d₅₀ [μm] | | | | | |
| of fine portion | | | | | |
| PSD after | 19 | 18 | 15 | 17 | 12 |
| 5′MS: d₅₀ [μm] | | | | | |
| of coarse | | | | | |
| portion | | | | | |
| PSD after | 62/3.0 | 65/3.0 | 63/1.6 | 67/<1 | 78/<1 |
| 5′MS + 10′US: | | | | | |
| quantity [%]/ | | | | | |
| d₅₀ [μm] of | | | | | |
| fine portion | | | | | |
| PSD after | 16 | 13 | 15 | 14 | 11 |
| 5′MS + 10′US: | | | | | |
| d₅₀ [μm] of | | | | | |
| coarse portion | | | | | |

Table 3b: Additionally calcined for 1 h at 600° C.:

| | | | | | |
|---|---|---|---|---|---|
| Weight loss | 8.5 | 7.0 | 5.6 | 4.0 | 3.4 |
| [%] | | | | | |
| BET (thereof | 102 (0) | 139 (0) | 138 (0) | 128 (0) | 122 (0) |
| in micropores) | | | | | |
| [m²/g] | | | | | |
| Pore volume | 0.34 (0.34) | 0.38 (0.38) | 0.34 (0.34) | 0.35 (0.35) | 0.37 (0.37) |
| (thereof in | | | | | |
| mesopores | | | | | |
| [cm³/g] | | | | | |
| Crystalline | Anatase, η- | Anatase, | Anatase | Anatase | Anatase |
| compounds | or γ-Al₂O₃ | a little η- or | | | |
| found | | γ-Al₂O₃ | | | |
| Anatase | 9 | 10 | 10 | 10 | 12 |
| crystallite size | | | | | |
| [nm] | | | | | |

TABLE 4
Products of TiO₂/Al₂O₃ of different compositions after the HT
process, distribution of mesopore diameter as a function of the
temperature of HT treatment (Example 4); d₅₀ in nm/±σ without
dimension:

| | Mole % Al, | | | | |
|---|---|---|---|---|---|
| | remainder Ti | | | | |
| | 40 | 25 | 15 | 7 | 3 |
| After drying | 2.7/0.38 | 2.7/0.37 | 2.7/0.37 | 2.6/0.36 | 2.7/0.38 |
| HT treated 4 h | 5.5/0.20 | 5.0/0.21 | 4.7/0.19 | 5.9/0.23 | |
| at 120° C. | | | | | |
| HT treated 4 h | 7.0/0.24 | 6.2/0.23 | 6.4/0.22 | 7.1/0.23 | |
| at 150° C. | | | | | |
| HT treated 4 h | 8.5/0.28 | 9.5/0.29 | 8.5/0.26 | 10.0/0.26 | 11.5/0.25 |
| at 180° C. | | | | | |

TABLE 5
Product of TiO₂/Al₂O₃ with 40 mole % Al, remainder Ti, effect of
duration of HT treatment at 150° C. (Example 4):

| | Duration in h | | | | |
|---|---|---|---|---|---|
| | 0 | 2 | 4 | 8 | 16 |
| BET (thereof | 415 (76) | 243 (0) | 198 (0) | 203 (0) | 158 (0) |
| in micropores) | | | | | |
| [m²/g] | | | | | |
| Pore volume | 0.29 | 0.39 | 0.37 | 0.37 | 0.38 |
| [cm³/g] | | | | | |
| Mesopores: | 2.7 | 5.8 | 7.0 | 6.8 | 8.8 |
| d₅₀ [nm] | | | | | |
| Crystalline | — | | | | Anatase, |
| compounds | (amorphous) | | | | boehmite |
| found | | | | | |
| Anatase | | | | | 9 |
| crystallite size | | | | | |
| [nm] | | | | | |

TABLE 6
Products of TiO₂/Al₂O₃ with 25 mole % Al, remainder Ti: effect of
the concentrations of the Ti and Al solutions used for precipitation
(Examples 5 and 6):

| | |
|---|---|
| Example 5 | Example 6 |

Table 6a: After drying:

| | | |
|---|---|---|
| % NH₄ | 1.1 | 0.9 |
| ppm Na | 260 | 340 |
| % SO₄ | 1.1 | 1.3 |
| BET (thereof | 329 (132) | 338 (143) |
| in micropores) | | |
| [m²/g] | | |
| Pore volume | 0.32 (0.28) | 0.29 (0.22) |
| (thereof in | | |
| mesopores) | | |
| [cm³/g] | | |
| Mesopores: | 3.3/0.44 | 4.1/0.42 |
| d₅₀ [nm]/ | | |
| ±σ [—] | | |
| Crystalline | - (amorphous) | - (amorphous) |
| compounds | | |
| found | | |

Table 6b: Calcined at 450° C.:

| | | |
|---|---|---|
| BET (thereof | 237 (0) | 214 (0) |
| in micropores) | | |
| [m²/g] | | |
| Pore volume | 0.26 (0.27) | 0.23 (0.22) |
| (thereof in | | |
| mesopores | | |
| [cm³/g] | | |
| Mesopores: | 4.6/0.18 | 5.8/0.23 |
| d₅₀ [nm]/ | | |
| ±σ [—] | | |
| Crystalline | - (amorphous) | Anatase |
| compounds | | |
| found | | |
| Anatase | | 6 |
| crystallite size | | |
| [nm] | | |

Table 6c: Calcined at 600° C.:

| | | |
|---|---|---|
| BET (thereof | 136 (0) | 119 (0) |
| in micropores) | | |
| [m²/g] | | |
| Pore volume | 0.19 (0.20) | 0.17 (0.15) |
| (thereof in | | |
| mesopores | | |
| [cm³/g] | | |
| Mesopores: | 7.4/0.19 | 8.6/0.22 |
| d₅₀ [nm]/ | | |
| ±σ [—] | | |
| Crystalline | Anatase | Anatase |
| compounds | | |
| found | | |
| Anatase | 8 | 10 |
| crystallite size | | |
| [nm] | | |

TABLE 7
Products of TiO₂/Al₂O₃ of different compositions after the calcining
process (Example 1):

| Mole % Al, remainder Ti | | | | |
|---|---|---|---|---|
| 40 | 25 | 15 | 7 | 3 |

Table 7a: After drying:

| | | | | | |
|---|---|---|---|---|---|
| ppm Na | 2000 | 2100 | 5900 | 7500 | 1300 |
| % SO₄ | 0.09 | 0.03 | 0.03 | 0.03 | 0.03 |
| BET (thereof | 406 (220) | 424 (279) | 433 (310) | 410 (270) | 389 (240) |
| in micropores) | | | | | |
| [m²/g] | | | | | |
| Pore volume | 0.61 (0.46) | 0.64 (0.47) | 0.54 (0.36) | 0.68 (0.52) | 0.88 (0.75) |
| (thereof in | | | | | |
| mesopores) | | | | | |
| [cm³/g] | | | | | |
| Crystalline | — | — | — | — | — |
| compounds | (amorphous) | (amorphous) | (amorphous) | (amorphous) | (amorphous) |
| found | | | | | |

Table 7b: Calcined at 450° C.:

| | | | | | |
|---|---|---|---|---|---|
| Weight loss | 22.1 | 15.1 | 15.6 | 19.9 | 16.1 |
| [%] | | | | | |
| BET (thereof | 274 (0) | 249 (0) | 235 (20) | 72 (0) | 67 (5) |
| in micropores) | | | | | |
| [m²/g] | | | | | |
| Pore volume | 0.50 | 0.65 | 0.75 (0.71) | 0.52 | 0.66 |
| [cm³/g] | | | | | |
| Crystalline | — | — | — | Anatase | Anatase |
| compounds | (amorphous) | (amorphous) | (amorphous) | | |
| found | | | | | |
| Anatase | | | | 15 | 16 |
| crystallite size | | | | | |
| [nm] | | | | | |
| PSD after | 50/2.1 | | | | |
| 5′MS: quantity | | | | | |
| [%]/d₅₀ [μm] | | | | | |
| of fine portion | | | | | |
| PSD after | 30 | | | | |
| 5′MS: d₅₀ [μm] | | | | | |
| of coarse | | | | | |
| portion | | | | | |
| PSD after | 70/2.1 | | | | |
| 5′MS + 10′US: | | | | | |
| quantity [%]/ | | | | | |
| d₅₀ [μm] of | | | | | |
| fine portion | | | | | |
| PSD after | 14 | | | | |
| 5′MS + 10′US: | | | | | |
| d₅₀ [μm] of | | | | | |
| coarse portion | | | | | |

Table 7c: Calcined at 600° C.:

| | | | | | |
|---|---|---|---|---|---|
| Weight loss | 24.0 | 16.7 | 15.8 | 19.9 | 16.5 |
| [%] | | | | | |
| BET (thereof | 164 (0) | 98 (0) | 80 (0) | 61 (3) | 53 (6) |
| in micropores) | | | | | |
| [m²/g] | | | | | |
| Pore volume | 0.45 | 0.48 | 0.48 | 0.64 | 0.55 |
| [cm³/g] | | | | | |
| Crystalline | Anatase | Anatase | Anatase | Anatase | Anatase |
| compounds | | | | | |
| found | | | | | |
| Anatase | 11 | 11 | 13 | 16 | 18 |
| crystallite size | | | | | |
| [nm] | | | | | |
| PSD after | 54/2.0 | 40/1.8 | 40/1.8 | 27/2.0 | 17/2.0 |
| 5′MS: quantity | | | | | |
| [%]/d₅₀ [μm] | | | | | |
| of fine portion | | | | | |
| PSD after | 28 | 42 | 53 | 42 | 42 |
| 5′MS: d₅₀ [μm] | | | | | |
| of coarse | | | | | |
| portion | | | | | |
| PSD after | 80/2.1 | 67/1.9 | 70/1.9 | 50/1.9 | 43/2.0 |
| 5′MS + 10′US: | | | | | |
| quantity [%]/ | | | | | |
| d₅₀ [μm] of | | | | | |
| fine portion | | | | | |
| PSD after | 15 | 17 | 13 | 21 | 21 |
| 5′MS + 10′US: | | | | | |
| d₅₀ [μm] of | | | | | |
| coarse portion | | | | | |

Table 7d: Calcined at 800° C.:

| | | | | | |
|---|---|---|---|---|---|
| Weight loss | 23.8 | 16.7 | 15.9 | 19.7 | 19.7 |
| [%] | | | | | |
| BET (thereof | 76 (5) | 49 (6) | 35 | 33 | 23 |
| in micropores) | | | | | |
| [m²/g] | | | | | |
| Pore volume | 0.33 | 0.36 | | | |
| [cm³/g] | | | | | |
| Crystalline | Anatase | Anatase | Anatase | Anatase | Anatase, |
| compounds | | | | | rutile |
| found | | | | | |
| Anatase | 17 | 18 | 24 | 25 | |
| crystallite size | | | | | |
| [nm] | | | | | |
| PSD after | 57/1.9 | | | | |
| 5′MS: quantity | | | | | |
| [%]/d₅₀ [μm] | | | | | |
| of fine portion | | | | | |
| PSD after | 26 | | | | |
| 5′MS: d₅₀ [μm] | | | | | |
| of coarse | | | | | |
| portion | | | | | |
| PSD after | 66/2.0 | | | | |
| 5′MS + 10′US: | | | | | |
| quantity [%]/ | | | | | |
| d₅₀ [μm] of | | | | | |
| fine portion | | | | | |
| PSD after | 16 | | | | |
| 5′MS + 10′US: | | | | | |
| d₅₀ [μm] of | | | | | |
| coarse portion | | | | | |

1-25. (canceled)

26. An oxide mixture containing particles of Al₂O₃ and TiO₂, wherein it contains at least one solid solution of Al₂O₃ and TiO₂.

27. An oxide mixture according to claim 25, wherein the particles in the oxide mixture contain pores.

28. An oxide mixture according to claim 25, wherein the oxide mixture:
i contains 0.5 to 45 mole % Al;
ii contains at least one solid solution of Al₂O₃ and TiO₂ in one another; and
iii possesses pores with a pore diameter d₅₀ of between 2 and 50 nm.

29. An oxide mixture according to claim 25, wherein the logarithmic diameter distribution of the pores has a width a of no more than 0.50.

30. An oxide mixture according to claim 25, wherein the BET specific surface area of the oxide mixture is between 5 and 800 m²/g.

31. An oxide mixture according to claim 25, wherein the pore volume of the oxide mixture is between 0.02 and 2 cm³/g.

32. An oxide mixture according to claim 25, wherein 30% to 99 of the pore volume of the oxide mixture is formed by mesopores.

33. An oxide mixture according to claim 25, wherein 100% of the pore volume of the oxide mixture is formed by mesopores.

34. An oxide mixture according to claim 25, wherein the pore size is adjustable.

35. An oxide mixture according to claim 25, wherein the oxide mixture contains amorphous TiO₂ with Al₂O₃ dissolved therein.

36. An oxide mixture according to claim 25, wherein the oxide mixture contains anatase crystals with Al₂O₃ dissolved therein.

37. An oxide mixture according to claim 25, wherein the oxide mixture contains anatase crystals intergrown with an amorphous solid solution of Al₂O₃ and TiO₂ in one another.

38. An oxide mixture according to claim 25, wherein the particles in the oxide mixture have pores with a pore diameter d₅₀ of between 1 and 2 nm.

39. An oxide mixture according to claim 25, wherein the particle size d₅₀ of the solid solution is from 0.5 to 10 μm.

40. A process for the production of an oxide mixture according to claim 25, comprising slowly adding an aqueous Ti solution to a precharged alkaline Al solution with the addition of a base to provide a pH of from 4 to 8, and precipitating hydrated preforms of TiO₂ and Al₂O₃ from the resultant aqueous solution, without addition of organic or organometallic compounds.

41. A process for the production of an oxide mixture according to claim 25, comprising slowly adding an alkaline Al solution to a Ti solution and adding a base to yield a pH of from 4 to 8 to precipitate hydrated preforms of TiO₂ and Al₂O₃, without addition of organic or organometallic compounds.

42. A production process according to claim 40, wherein the aqueous Ti solution is an aqueous titanyl chloride, nitrate or sulfate solution which contains between 50 and 400 g/l, preferably between 60 and 300 g/l, particularly preferably between 60 and 250 g/l titanium.

43. A process according to claim 40, wherein the aqueous Al solution is an aqueous alkali aluminate solution which contains between 50 and 350 g/l aluminum.

44. A process according to claim 40, wherein the oxide mixture is separated off as a precipitation product.

45. A process according to claim 40, wherein the purified and dried oxide mixture is calcined at temperatures of between 350 and 900° C. over a period of between 1 and 20 hours.

46. A process according to claim 40, wherein the oxide mixture is HT-treated at temperatures of 100 to 250° C. over a period of between 1 and 20 hours.

47. An oxide mixture prepared by the production process of claim 40.

48. A method comprising catalyzing a chemical reaction with the oxide mixture according to claim 25.

49. A chromatographic column comprising the oxide mixture of claim 25.

50. The method of claim 48, both as a catalyst and/or catalyst support and as a chromatographic solid phase in one and the same apparatus in an industrial production process.

51. The method of claim 48, where in the process is selected from the group consisting of (hydro)cracking or desulfurization of naphtha and heavy oil, a fluid-catalytic cracking process, a deep-catalytic process for high-sulfur raw material, an hydrodesulfurization process, hydrogenation of aromatics, isomerization or dehydrogenation of low-boiling hydrocarbon, a petroleum process to yield olefin, a butane and butene isomerisation process, a Fischer-Tropsch synthesis, coal liquefaction, partial oxidation of saturated and unsaturated hydrocarbons, formation of acetic acid, acrylic acid, maleic acid, phthalic or terephthalic acid, epoxidation of long-chain olefins, terpenes or cyclohexane, and hydroxylation of polynuclear aromatics.
